(19) United States
(12) Patent Application Publication
(10) Pub. No.: US 2009/0093539 A1
(43) Pub. Date: Apr. 9, 2009

(54) ANTI-ANGIOGENIC COMPOSITIONS AND METHODS OF USE THEREOF

(76) Inventor: Kin-Ping WONG, Fresno, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/868,349

(22) Filed: Oct. 5, 2007

(51) Int. Cl. A61K 31/343; A61P 35/00; A61P 19/02; A61P 27/02

(52) U.S. Cl. 514/468

(57) ABSTRACT

The present invention provides methods of reducing angiogenesis in an individual. The methods generally involve administering to the individual an effective amount of a tanshinone compound. The methods are useful to treat conditions associated with or resulting from angiogenesis, particularly pathological angiogenesis. The invention further provides methods of treating a condition associated with or resulting from angiogenesis.

### BACKGROUND

Angiogenesis and vasculogenesis are processes involved in the growth of blood vessels. Angiogenesis is the process by which new blood vessels are formed from extant capillaries, while vasculogenesis involves the growth of vessels deriving from endothelial progenitor cells.

Both angiogenesis and vasculogenesis involve the proliferation of endothelial cells. Endothelial cells line the walls of blood vessels; capillaries are comprised almost entirely of endothelial cells. The angiogenic process involves not only increased endothelial cell proliferation, but also comprises a cascade of additional events, including protease secretion by endothelial cells, degradation of the basement membrane, migration through the surrounding matrix, proliferation, alignment, differentiation into tube-like structures, and synthesis of a new basement membrane. Vasculogenesis involves recruitment and differentiation of mesenchymal cells into angioblasts, which then differentiation into endothelial cells which then from de novo vessels.

Angiogenesis and vasculogenesis, and the factors that regulate these processes, are important in embryonic development, inflammation, and wound healing. Inappropriate, or pathological, angiogenesis is involved in the growth of atherosclerotic plaque, diabetic retinopathy, degenerative maculopathy, retrolental fibroplasia, idiopathic pulmonary fibrosis, acute adult respiratory distress syndrome, and asthma. Furthermore, tumor progression is associated with neovascularization, which provides a mechanism by which nutrients are delivered to the progressively growing tumor tissue.

There is a need in the art for methods of reducing pathological angiogenesis.

### SUMMARY OF THE INVENTION

The present invention provides methods of reducing angiogenesis in an individual. The methods generally involve administering to the individual an effective amount of a tanshinone compound. The methods are useful to treat conditions associated with or resulting from angiogenesis, particularly pathological angiogenesis. The invention further provides methods of treating a condition associated with or resulting from angiogenesis.

### BRIEF DESCRIPTION OF THE DRAWINGS

FIG. 1 depicts inhibition of endothelial cell proliferation by 15,16-dihydrotanshinone.

### DEFINITIONS

The terms “treatment,” “treating,” and the like are used herein to generally mean obtaining a desired pharmacologic and/or physiologic effect, e.g., reduction of angiogenesis and/or vasculogenesis. The effect may be prophylactic in terms of completely or partially preventing a disease or symptom thereof and/or may be therapeutic in terms of a partial or complete cure for a disease and/or adverse effect attributable to the disease “Treatment” as used herein covers any treatment of a disease in a mammal, e.g., a human, and includes: (a) preventing a disease or condition from occurring in a subject who may be predisposed to the disease but has not yet been diagnosed as having it; (b) inhibiting the disease, e.g., arresting its development; or (c) relieving the disease, where inhibiting the disease and relieving the disease is in an individual diagnosed with the disease (e.g., due to the presence of one or more disease symptoms). In the context of the present invention, reduction of angiogenesis and/or vasculogenesis is employed for subject having a disease or condition amenable to treatment by reducing angiogenesis.

By “therapeutically effective amount of a tanshinone compound” is meant an amount of a tanshinone compound effective to facilitate a desired therapeutic effect, e.g., a desired reduction of angiogenesis and/or vasculogenesis. The precise desired therapeutic effect will vary according to the condition to be treated.

By “isolated” is meant that the compound is separated from all or some of the components that accompany it in nature.

The term “individual” is used interchangeably herein with “host,” “subject,” and “patient” to refer to a vertebrate, e.g., a mammal, including non-human primates, humans, farm mammals (e.g., cows, pigs, goats, horses, sheep, and other ungulates), sport animals (e.g., horses), rodents (e.g., mice, rats), lagomorphs, and mammalian pets (e.g., cats, dogs, etc.).

By “substantially pure tanshinone compound” is meant that the tanshinone compound has been separated from components that accompany it in nature. For example, a tanshinone compound is substantially pure when it is at least 60%, at least 75%, at least 80%, at least 90%, at least 95%, or at least 99%, by weight, free from naturally-occurring organic molecules with which it is naturally associated. A substantially pure tanshinone compound can be obtained, for example, by extraction from a natural source, by chemically synthesizing the compound, or by a combination of purification and chemical modification. Purity can be measured by any appropriate method, e.g., chromatography, mass spectroscopy, high performance liquid chromatography, gas chromatography/mass spectrometry analysis, etc.

“Tanshinone compound” as used herein refers to any tanshinone compound of Formula I, as provided below, with 15,16-dihydrotanshinone being of particular interest. The term “tanshinone compound” also includes metabolites of a tanshinone compound.

As used herein, “pharmaceutically acceptable derivatives” of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

The term “lower alkyl” as used herein refers to C1 to C8 alkyl, e.g., C1 to C6 alkyl, C1 to C4 alkyl, and the like, which may be linear or branched, and which may be saturated or unsaturated.

The term “cycloalkyl” refers to a mono-, bi-, or tricyclic saturated ring that is fully saturated or partially unsaturated. Examples of such a group included cyclopropyl, cyclobutyl, cyclopentyl, cyclohexyl, cycloheptyl, adamantyl, cyclooctyl, cis- or trans decalin, bicyclo[2.2.1]hept-2-ene, cyclohex-1-enyl, cyclopent-1-enyl, 1,4-cyclooctadienyl, and the like. “Cycloalkyl” is specified as such herein, and is in some embodiments C3, C4 or C5 to C6 or C8 cycloalkyl.

“Lower alkenyl” as used herein refers to C1 to C4 alkenyl; and “lower alkoxy” as used herein refers to C1 to C4 alkoxy.

“Alkoxy” as used herein refers to linear or branched, saturated or unsaturated oxo-hydrocarbon chains, including for example methoxy, ethoxy, propoxy, isopropoxy, butoxy, and t-butoxy.

“Alkylogen” as used herein refers to alkyl or loweralkyl in which one, two, three or more (e.g., all) hydrogens thereon have been replaced with halo. Examples of alkylogen include but are not limited to trifluoromethyl, chloromethyl, 2-chloroethyl, 2-bromoethyl, and 2-iodoethyl. Alkylogens may also be referred to as haloalkyl or perhaloalkyl (e.g. fluoroalkyl; perfluoroalkyl).

The term “aryl” as used herein refers to C3 to C10 cyclic aromatic groups such as phenyl, naphthyl, and the like, and includes substituted aryl groups such as tolyl.

“Halo” as used herein refers to any halogen group, such as chloro, fluoro, bromo, or iodo.

The term “hydroxyalkyl” as used herein refers to C1 to C4 linear or branched hydroxy-substituted alkyl, i.e., —CH₂OH, —(CH₂)₂OH, etc.

The term “aminoalkyl” as used herein refers to C1 to C4 linear or branched amino-substituted alkyl, wherein the term “amino” refers to the group NR′R″, wherein R′ and R″ are independently selected from H or lower alkyl as defined above, e.g. —NH₂, —NHCH₃, —N(CH₃)₂, etc.

The term “oxyalkyl” as used herein refers to C1 to C4 oxygen-substituted alkyl, i.e., —OCH₃, and the term “oxyaryl” as used herein refers to C3 to C10 oxygen-substituted cyclic aromatic groups.

The term “alkylenedioxy” refers to a group of the general formula—ORNO—, —ORNORN—, or —RNORNORN—where each RN is independently alkyl.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a tanshinone compound” includes a plurality of such compound and reference to “the tanshinone formulation” includes reference to one or more tanshinone formulations and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present invention provides methods of reducing angiogenesis in an individual. The methods generally involve administering to the individual an effective amount of a tanshinone compound. The methods are useful to treat conditions associated with or resulting from angiogenesis, particularly pathological angiogenesis. The invention further provides methods of treating a condition associated with or resulting from angiogenesis.

The inventors have observed that a tanshinone compound, 15,16-dihydrotanshinone (also known as “15,16-dihydrotanshinone I”), inhibits endothelial cell proliferation. The results presented herein indicate that tanshinone compounds are useful to treat conditions and disorders associated with and/or resulting from pathological angiogenesis, including, e.g., cancer metastasis, atherosclerosis, proliferative retinopathies, excessive fibrovascular proliferation as seen with chronic arthritis, psoriasis, and vascular malformations such as hemangiomas.

### Methods of Reducing Pathological Angiogenesis

The present invention provides methods of reducing angiogenesis in an individual. The methods generally involve administering to an individual in need thereof an effective amount of a tanshinone compound. In some embodiments, a subject method involves administering a pharmaceutical formulation comprising an isolated, purified tanshinone compound.

### Tanshinone Compounds

Suitable tanshinone compounds include those of Formula I:

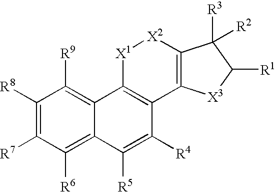

wherein

R¹, R², R³, R⁴, R⁵, R⁶, R⁷, R⁸, and R⁹ are each independently selected from H, lower alkyl, hydroxy, lower alkoxy, halo, amino, aminoalkyl, nitro, heteroaryl, aryl, OC(═O)R¹⁴, OC(═O)OR¹⁴, OC(═O)N(R¹⁴)₂, O(CH₂)ₘN(R¹⁴)₂, C(═O)N(R¹⁴)₂, and O(CH₂)ₘCOOH, where m is 1-5 and R¹⁴ is H or lower alkyl;

or R¹ and R² together form a covalent bond;

or R² and R³ together form =Z, where Z is selected from O, S, and NH;

X¹ and X² are each independently selected from—C(R¹⁵)(R¹⁶)—, O, S, NH, C═O, C═S, C═.NH, SO, and SO₂, wherein R¹⁵ and R¹⁶ are each independently selected from H, lower alkyl, hydroxy, lower alkoxy, halo, amino, aminoalkyl, nitro, heteroaryl, aryl, OC(═O)R¹⁷, OC(═O)OR¹⁷, OC(═O)N(R¹⁷)₂, O(CH₂)ₘN(R¹⁷)₂2, C(═O)N(R¹⁷)₂, and O(CH₂)ₘCOOH, where m is 1-5 and R¹⁷ is H or lower alkyl;

or X¹ and X² together form —C═C—; and

X³ is selected from O, S, NH, or (CH₂)ₚ, where p is 1-3;

or a pharmaceutically acceptable salt thereof.

Where the compound is a dihydrotanshinone, the compound is of the formula:

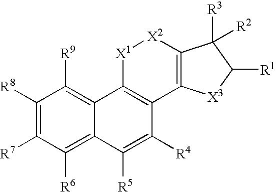

wherein

R¹, R², R³, R⁴, R⁵, R⁶, R⁷, R⁸, and R⁹ are each independently selected from H, lower alkyl, hydroxy, lower alkoxy, halo, amino, aminoalkyl, nitro, heteroaryl, aryl, OC(═O)R¹⁴, OC(═O)OR¹⁴, OC(═O)N(R¹⁴)₂, O(CH₂)ₘN(R¹⁴)₂, C(═O)N(R¹⁴)₂, and O(CH₂)ₘCOOH, where m is 1-5 and R¹⁴ is H or lower alkyl, with R¹, R², R³, R⁴, R⁵, R⁶, R⁷, R⁸, and R⁹ being independently selected from H or a lower alkyl being of particular interest;

X¹ and X² are each independently selected from C═O, C═S, C═.NH, SO, and SO₂, with C═O being of particular interest; and

X³ is selected from O, S, NH, and (CH₂)ₚ, where p is 1-3, with O, S or NH being of particular interest;

or a pharmaceutically acceptable salt thereof.

In some embodiments, the compound is a dihydrotanshinone compound of the formula:

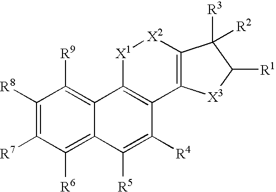

wherein

R¹, R², R³, R⁴, R⁵, R⁶, R⁷, R⁸, and R⁹ are each independently selected from H, lower alkyl, hydroxy, lower alkoxy, halo, amino, aminoalkyl, nitro, heteroaryl, aryl, OC(═O)R¹⁴, OC(═O)OR¹⁴, OC(═O)N(R¹⁴)₂, O(CH₂)ₘN(R¹⁴)₂, C(═O)N(R¹⁴)₂, and O(CH₂)ₘCOOH, where m is 1-5 and R¹⁴ is H or lower alkyl, with R¹, R², R³, R⁴, R⁵, R⁶, R⁷, R⁸, and R⁹ being independently selected from H or a lower alkyl being of particular interest;

X¹ and X² are C═O; and

X³ is O;

or a pharmaceutically acceptable salt thereof.

Synthesis of a variety of compounds of Formula I is described in, e.g., U.S. Patent Publication No. 2005/0250751; and Danheiser et al. (1992) Tetrahedron Lett. 33:1149. Tanshinone compounds of Formula I can also be isolated from a naturally occurring source, e.g., from the root of Salvia miltiorrhiza Bunge.

In some embodiments, a suitable compound is 15,16-dihydrotanshinone (also known as “Dihydrotanshinone I”), which has the structure:

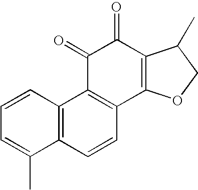

15,16-dihydrotanshinone can be isolated from a naturally-occurring source of the compound. Purification can be carried out, e.g., using a method as described in Aoki et 1. (1979) Anal. Biochem. 95:575-578). 15,16-dihydrotanshinone can also be chemically synthesized in vitro. Synthesis of 15,16-dihydrotanshinone is carried out using any known method, e.g., a method as described in Danheiser et al. (1992) Tetrahedron Lett. 33:1149.

A pharmaceutically acceptable salt of a tanshinone compound can be administered. Pharmaceutically acceptable salts of a tanshinone compound are salts that retain the desired biological activity of the parent tanshinone compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine.

Exemplary salts of interest include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylproprionate, picrate, pivalate, propionate, succinate, tartrate, thiocyanate, tosylate and undecanoate salts. Other examples of salts include anions of a tanshinone compound compounded with a suitable cation such as Na⁺, NH₄⁺, and NW₄⁺ (wherein W is a C₁₋₄ alkyl group).

Suitable tanshinone compounds can be identified using readily available methods. The ability of a tanshinone compound to reduce angiogenesis can be assessed in vitro or in vivo using any known method, including, but not limited to, an in vitro endothelial cell assay, a Matrigel assay, a disc angiogenesis system, a chick embryonic chorioallantoic membrane assay, and the like.

In some embodiments, a tanshinone compound, e.g., a dihydrotanshinone compound, that is administered to an individual in a subject method is purified, e.g., the tanshinone compound is at least 80% pure, at least 85% pure, at least 90% pure, at least 95% pure, at least 98% pure, or at least 99% pure.

### Conditions Amenable to Treatment

Any condition or disorder that is associated with or that results from pathological angiogenesis, or that is facilitated by neovascularization (e.g., a tumor that is dependent upon neovascularization), is amenable to treatment with a tanshinone compound.

Conditions and disorders amenable to treatment (which may be referred to herein as “angiogenesis-related disorders” or “angiogenesis-associated disorders”) include, but are not limited to, cancer; atherosclerosis; proliferative retinopathies such as diabetic retinopathy, age-related maculopathy (e.g., age-related macular degeneration), vitreoretinopathy, retrolental fibroplasia; excessive fibrovascular proliferation as seen with chronic arthritis; psoriasis; and vascular malformations such as hemangiomas, and the like.

The instant methods are useful in the treatment of both primary and metastatic solid tumors, particularly metastatic tumors, including carcinomas, sarcomas, leukemias, and lymphomas. Of particular interest is the treatment of tumors occurring at a site of angiogenesis. In some embodiments, the methods provide for reduction of metastasis of a cancer cell from a solid tumor. Thus, the methods are useful in the treatment of any neoplasm, including, but not limited to, carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract, (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). The instant methods are also useful for treating solid tumors arising from hematopoietic malignancies such as leukemias (i.e. chloromas, plasmacytomas and the plaques and tumors of mycosis fungoides and cutaneous T-cell lymphoma/leukemia) as well as in the treatment of lymphomas (both Hodgkin's and non-Hodgkin's lymphomas). In addition, the instant methods are useful for reducing metastases from the tumors described above either when used alone or in combination with radiotherapy and/or other chemotherapeutic agents.

Other conditions and disorders amenable to treatment using the methods of the instant invention include autoimmune diseases such as rheumatoid, immune and degenerative arthritis; various ocular diseases such as diabetic retinopathy, retinopathy of prematurity, corneal graft rejection, retrolental fibroplasia, neovascular glaucoma, rubeosis, retinal neovascularization due to macular degeneration, hypoxia, angiogenesis in the eye associated with infection or surgical intervention, and other abnormal neovascularization conditions of the eye; skin diseases such as psoriasis; blood vessel diseases such as hemangiomas, and capillary proliferation within atherosclerotic plaques; Osler-Webber Syndrome; plaque neovascularization; telangiectasia; hemophiliac joints; angiofibroma; and excessive wound granulation (keloids).

### Reducing Pathological Angiogenesis In Vivo

The instant invention provides a method of reducing angiogenesis in a mammal. The method generally involves administering to a mammal in need thereof a tanshinone compound in an amount effective to reduce angiogenesis. An effective amount of a tanshinone compound reduces angiogenesis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or more, when compared to an untreated (e.g., a placebo-treated) control.

Whether angiogenesis is reduced can be determined using any known method. Methods of determining an effect of an agent (e.g., a tanshinone compound) on angiogenesis are known in the art and include, but are not limited to, inhibition of neovascularization into implants impregnated with an angiogenic factor; inhibition of blood vessel growth in the cornea or anterior eye chamber; inhibition of endothelial cell proliferation, migration or tube formation in vitro; the chick chorioallantoic membrane assay; the hamster cheek pouch assay; the polyvinyl alcohol sponge disk assay. Such assays are well known in the art and have been described in numerous publications, including, e.g., Auerbach et al. ((1991) Pharmac. Ther. 51: 1-11), and references cited therein.

The invention further provides methods for treating a condition or disorder associated with or resulting from pathological angiogenesis. In the context of ocular disorders associated with or resulting from pathological angiogenesis, a reduction in pathological angiogenesis according to the methods of the invention effects an improvement in vision, or at least maintenance of vision, or a reduction in vision loss. In the context of cancer therapy, a reduction in angiogenesis according to the methods of the invention effects a reduction in tumor metastasis. Whether metastasis is reduced can be determined using any known method, e.g., imaging methods using dyes, computed tomography (CT), and magnetic resonance imaging (MRI). In the context of arthritis, a reduction in pathological angiogenesis according to the methods of the invention effects a reduction in pain associated with arthritis (e.g., joint pain), and/or a reduction in joint swelling, and/or an increase in patient mobility, and/or an increase in joint mobility.

In the treatment of an angiogenesis-associated disorder, a tanshinone compound is administered in an amount of from about 5 μg to about 1000 mg per dose, e.g., from about 5 μg to about 50 μg, from about 50 μg to about 100 μg, from about 100 μg to about 500 μg, from about 500 μg to about 1 mg, from about 1 mg to about 10 mg, from about 10 mg to about 20 mg, from about 20 mg to about 25 mg, from about 25 mg to about 50 mg, from about 50 mg to about 75 mg, from about 75 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 750 mg, or from about 750 mg to about 1000 mg per dose.

In some embodiments, the amount of a tanshinone compound per dose is determined on a per body weight basis. For example, in some embodiments, a tanshinone compound is administered in an amount of from about 0.01 mg/kg to about 50 mg/kg, e.g., from about 0.01 mg/kg to about 0.05 mg/kg, from about 0.05 mg/kg to about 0.1 mg/kg, from about 0.1 mg/kg to about 0.5 mg/kg, from about 0.5 mg/kg to about 1 mg/kg, from about 1 mg/kg to about 2 mg/kg, from about 2 mg/kg to about 3 mg/kg, from about 3 mg/kg to about 5 mg/kg, from about 5 mg/kg to about 7 mg/kg, from about 7 mg/kg to about 10 mg/kg, from about 10 mg/kg to about 15 mg/kg, from about 15 mg/kg to about 20 mg/kg, from about 20 mg/kg to about 25 mg/kg, from about 25 mg/kg to about 30 mg/kg, from about 30 mg/kg to about 40 mg/kg, or from about 40 mg/kg to about 50 mg/kg per dose.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

In some embodiments, multiple doses of a tanshinone compound are administered. The frequency of administration of a tanshinone compound can vary depending on any of a variety of factors, e.g., severity of the symptoms, etc. For example, in some embodiments, a tanshinone compound is administered once every 6 months, once every 3 months, once every 2 months, once every 6 weeks, once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). In some embodiments, a tanshinone compound is administered continuously.

The duration of administration of a tanshinone compound, e.g., the period of time over which a tanshinone compound is administered, can vary, depending on any of a variety of factors, e.g., patient response, etc. For example, a tanshinone compound can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, a tanshinone compound is administered for the lifetime of the individual.

In some embodiments, administration of a tanshinone compound is discontinuous, e.g., a tanshinone compound is administered for a first period of time and at a first dosing frequency; administration of the tanshinone compound is suspended for a period of time; then the tanshinone compound is administered for a second period of time for a second dosing frequency. The period of time during which administration of the tanshinone compound is suspended can vary depending on various factors, e.g., patient response; and will generally range from about 1 week to about 6 months, e.g., from about 1 week to about 2 weeks, from about 2 weeks to about 4 weeks, from about one month to about 2 months, from about 2 months to about 4 months, or from about 4 months to about 6 months, or longer. The first period of time may be the same or different than the second period of time; and the first dosing frequency may be the same or different than the second dosing frequency.

### Ocular Disorders

The present invention provides methods of treating various angiogenesis-associated ocular disorders, e.g., diabetic retinopathy, age-related macular degeneration, and the like. In some embodiments, the present invention provides methods of treating diabetic retinopathy. In some embodiments, the present invention provides methods of treating age-related macular degeneration (AMD), including neovascular (“wet”) AMD and dry AMD. In some embodiments, the present invention provides methods of treating neovascular AMD. The methods generally involve administering to an individual in need thereof (e.g., an individual diagnosed as having an angiogenesis-associated ocular disorder, e.g., diabetic retinopathy, AMD, etc.) a formulation comprising a tanshinone compound. Whether a given tanshinone compound, or a given dosage of a tanshinone compound, is effective in treating AMD can be determined by assessing the patient's vision.

In some embodiments, an “effective amount” of a tanshinone compound is an amount that, when administered in one or more doses, results in an at least 5%, at least 10%, at least 15%, at least 20%, at least 25%, at least 30%, at least 40%, at least 50%, or better, improvement in vision. In some embodiments, an “effective amount” of a tanshinone compound is an amount that, when administered in one or more doses, results in a slowing of deterioration of vision, e.g., maintenance of vision. In some embodiments, an “effective amount” of a tanshinone compound is an amount that, when administered in one or more doses, results in a loss of less than 15 letters, less than 10 letters, or less than 5 letters in visual acuity on the Early Treatment of Diabetic Retinopathy chart. In some embodiments, an “effective amount” of a tanshinone compound is an amount that, when administered in one or more doses, results in an improvement of more than 5 letters, more than 10 letters, or more than 15 letters (e.g., an improvement of from about 5 letters to about 10 letters, from about 10 letters to about 15 letters, or from about 15 letters to about 20 letters) in visual acuity on the Early Treatment of Diabetic Retinopathy chart.

In some embodiments, in the treatment of an angiogenesis-associated ocular disorder such as diabetic retinopathy or AMD, a tanshinone compound is administered by injection (e.g., intraocular (e.g., intravitreal), subcutaneous, or intravenous injection). Other suitable routes of administration include topical administration (to the eye, e.g., using eye drops), subconjunctival, perioocular, subtenon, retrobulbar (retro-orbital), or by iontophoretic delivery to the eye. In some embodiments, a tanshinone compound is administered orally.

### Arthritis

The present invention provides methods of treating arthritis, e.g. rheumatoid arthritis, osteoarthritis, psoriatic arthritis, and the like. The methods generally involve administering to an individual in need thereof (e.g., an individual who has been diagnosed with rheumatoid arthritis, osteoarthritis, psoriatic arthritis, etc) an effective amount of a tanshinone compound.

Whether a given tanshinone compound, or a given dosage of a tanshinone compound, is effective in treating arthritis can be determined by assessing patient joint pain, joint stiffness (e.g., patient mobility), joint swelling, etc.

In some embodiments, an “effective amount” of a tanshinone compound is an amount that, when administered in one or more doses, reduces joint swelling and/or joint pain by at least about 10%, at least about 20%, at least about 30%, at least about 40%, at least about 50%, at least about 60%, at least about 70%, at least about 80%, or at least about 90%, or more, compared to the level of joint swelling or joint pain in the absence of treatment with the tanshinone compound.

In the context of arthritis, a tanshinone compound can be administered by any of a number of routes of administration, including, e.g., intramuscular, subcutaneous, intravenous, and the like. In some embodiments, a tanshinone is administered systemically. In other embodiments, a tanshinone is administered locally, e.g., at or near a joint that is affected with arthritis. In some embodiments, a tanshinone compound is administered orally.

### Inhibition of Metastasis of Solid Tumors

The present invention provides methods of reducing metastasis of cancer cells from a solid tumor. The methods generally involve administering to an individual in need thereof (e.g., an individual who has a solid tumor and who is at risk that a cancer cell in the solid tumor will metastasize or in whom a cancer cell from a solid tumor has already metastasized) an effective amount of a tanshinone compound.

Whether a given tanshinone compound, or a given dosage of a tanshinone compound, is effective in reducing metastasis is determined using any of a number of imaging techniques, e.g., MRI, CT, and the like.

In some embodiments, an “effective amount” of a tanshinone compound is an amount that, when administered in one or more doses, reduces metastasis by at least about 10%, at least about 20%, at least about 30%, at least about 40%, at least about 50%, at least about 60%, at least about 70%, at least about 80%, or at least about 90%, or more, compared to the level of metastasis in the absence of treatment with the tanshinone compound.

### Combination Therapies

In some embodiments, a tanshinone compound is administered in a combination therapy with one or more other therapeutic agents, e.g., a cancer chemotherapeutic agent; VEGF antagonist; a TNF antagonist; an anti-inflammatory agent; and the like. A tanshinone compound (or analog, derivative, or pharmaceutically acceptable salt thereof) may be administered alone, or in conjunction with other substances and/or therapies, depending on the context of administration (i.e., desired end result, condition of the individual, and indications). “In conjunction with” means that an agent (e.g., tanshinone compound) is administered prior to, concurrently, or after other substance or therapy.

### Combination Therapies for Cancer Treatment

In some embodiments, a tanshinone compound is administered as an adjuvant therapy to a standard cancer therapy. Standard cancer therapies include surgery (e.g., surgical removal of cancerous tissue), radiation therapy, bone marrow transplantation, chemotherapeutic treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Cancer Chemotherapeutic Agents

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-1, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use in connection with the methods of the invention include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ (8) colony-stimulating factors; and (9) inhibitors of angiogenesis.

Combination Therapy with a VEGF Antagonist

In some embodiments, a tanshinone compound is administered in combination therapy with at least one additional anti-angiogenic factor. For example, in the reduction of pathological angiogenesis associated with cancer, in the treatment of an ocular disease that involves pathological angiogenesis (e.g., wet AMD), in the treatment of arthritis, etc., a tanshinone compound will in some embodiments be administered n combination therapy with at least one additional anti-angiogenic factor. Suitable anti-angiogenic factors include, e.g., vascular endothelial growth factor (VEGF) antagonists; TNP-470 (Yeh et al. (2000) Proc. Natl. Acad. Sci. USA 97:12782); and the like.

In some embodiments, a tanshinone compound is administered in combination therapy with a VEGF antagonist. Suitable VEGF antagonists include, but are not limited to, inhibitors of VEGFR1 tyrosine kinase activity; inhibitors of VEGFR2 tyrosine kinase activity; an antibody to VEGF; an antibody to VEGFR1; an antibody to VEGFR2; a ribozyme specific for VEGFR1 or VEGFR2; an antisense specific for VEGFR1 or VEGFR2; siRNA specific for VEGFR1 or VEGFR2; a soluble VEGFR; and the like.

Exemplary non-limiting VEGF antagonists that are suitable for use in a subject method include, but are not limited to, a compound as disclosed in U.S. Pat. No. 6,469,032 (e.g., 3-[(2,3-Dimethylpyrrol-5-yl)methylene]-2-indolinone, an inhibitor of VEGF-dependent phosphorylation of VEGFR-2 (known as “SU5416”; see also, Fong et al. (1999) Cancer Res. 59:99-106); 3-[2,4-dimethyl-5-(2-oxo-1,2-dihy-droindol-3-ylidenemethyl)-1H-pyrrol-3-yl]propionic acid (known as “SU6668”; an inhibitor of VEGFR-2; see, e.g., WO 99/61422; and Laird et al. (2000) Cancer Res. 60:4152); ZD4190, an inhibitor of VEGFR-1 and VEGFR-2 (see, e.g., Wedge et al. (2000) Cancer Res. 60:970; and Wedge et al. (2000) Adv. Exp. Med. Biol. 476:307-310); [N-(4-bromo-2-fluorophenyl)-6-methoxy-7-[(1-methylpiperidin-4-yl)methoxy]quinazolin-4-amine] (known as “ZD6474”; Wedge et al. (2002) Cancer Res. 62:4645); a 4-anilinoquinazoline compound as disclosed in Hennequin et al. ((2002) J. Med. Chem. 45(6):1300-12); Bevacizumab (Avastin™), a monoclonal antibody to VEGF; ZM323881 (Whittles et al. (2002) Microcirculation 9:513-522); PTK787/ZK22584, an inhibitor of VEGFR-1 and VEGFR-2 (Wood et al. (2000) Cancer Res. 60:2178-2189); Angiozyme™, an anti-VEGFR-1 ribozyme (Weng et al. (2001) Curr. Oncol. Rep. 3:141-146); a soluble VEGFR (see, e.g., Takayama et al. (2000) Cancer Res. 60:2169-2177; Mori et al. (2000) Gene Ther. 7:1027-1033; and Mahasreshti et al. (2001) Clin. Cancer Res. 7:2057-2066); a monoclonal antibody to VEGFR-2 (see, e.g., Prewett et al. (1999) Cancer Res. 59:5209-5218; Witte et al. (1998) Cancer Metastasis Rev. 17:155-161; Brekken et al. (2000) Cancer Res. 60:5117-5124; Kunkel et al. (2001) Cancer Res. 61:6624-6628); a compound as disclosed in any of U.S. Pat. Nos. 5,792,783, 5,834,504, 5,883,113, 5,883,116, 5,886,020, 6,225,335, 6,323,228, and 6,469,032; a soluble VEGFR as disclosed in U.S. Patent Publication No. 20030181377; a compound as disclosed in U.S. Patent Publication No. 20030176487; an antibody to VEGFR as disclosed in U.S. Patent Publication No. 20030175271; a compound as disclosed in U.S. Patent Publication No. 20030171378; and the like.

### Combination Therapies for Treatment of AMD

In some embodiments, a tanshinone compound is administered in conjunction with at least a second therapeutic agent suitable for treating AMD. Agents (other than a tanshinone compound) that are suitable for treating AMD include, but are not limited to, a VEGF antagonist as described above; Visudyne® (verteporfin) with photodynamic therapy (PDT); and the like. Current therapies for AMD with can be used in conjunction with treatment with a tanshinone compound include, Macugen (pegaptanib sodium); Visudyne® (verteporfin) with PDT; Avastin™ Becvacizumab; and Lucentis (ranibizumab), a humanized monoclonal antibody that binds VEGF-A.

### Combination Therapies for Treatment of Arthritis

In some embodiments, a tanshinone compound is administered in conjunction with at least a second therapeutic agent suitable for treating arthritis. Agents (other than a tanshinone compound) that are suitable for treating arthritis include, but are not limited to, tumor necrosis factor-α(TNF-α) antagonists (e.g., soluble TNFR ENBREL® etanercept; infliximab (REMICADE®, Centocor); adalimumab (HUMIRA™, Abbott)); methotrexate; corticosteroids; disease modifying anti-rheumatic drugs; non-steroidal anti-inflammatory agents; and the like.

ENBREL® is a dimeric fusion protein consisting of the extracellular ligand-binding portion of the human 75 kilodalton (p75) TNFR linked to the Fc portion of human IgG1. The Fc component of ENBREL® contains the CH2 domain, the CH3 domain and hinge region, but not the CH1 domain of IgG1. ENBREL® is produced in a Chinese hamster ovary (CHO) mammalian cell expression system. It consists of 934 amino acids and has an apparent molecular weight of approximately 150 kilodaltons. Smith et al. (1990) Science 248:1019-1023; Mohler et al. (1993) J. Immunol. 151:1548-1561; U.S. Pat. No. 5,395,760; and U.S. Pat. No. 5,605,690.

REMICADE® is a chimeric monoclonal anti-TNF-α antibody that includes about 25% mouse amino acid sequence and about 75% human amino acid sequence. REMICADE® comprises a variable region of a mouse monoclonal anti-TNF-α antibody fused to the constant region of a human IgG1. Elliott et al. (1993) Arthritis Rheum. 36:1681-1690; Elliott et al. (1994) Lancet 344:1105-1110; Baert et al. (1999) Gastroenterology 116:22-28. HUMIRA™ is a human, full-length IgG1 monoclonal antibody that was identified using phage display technology. Piascik (2003) J. Am. Pharm. Assoc. 43:327-328.

### Formulations, Doses, Routes of Administration

The present invention provides formulations comprising an amount of a tanshinone compound effective for reducing pathological angiogenesis. Compositions comprising a tanshinone compound are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Suitable tanshinone compounds include those of Formula I:

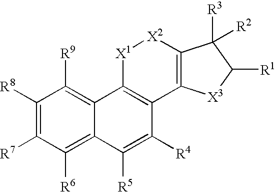

wherein

R¹, R², R³, R⁴, R⁵, R⁶, R⁷, R⁸, and ⁹ are each independently selected from H, lower alkyl, hydroxy, lower alkoxy, halo, amino, aminoalkyl, nitro, heteroaryl, aryl, OC(═O)R¹⁴, OC(═O)OR¹⁴, OC(═O)N(R¹⁴)₂, O(CH₂)ₘN(R¹⁴)₂, C(═O)N(R¹⁴)₂, and O(CH₂)ₘCOOH, where m is 1-5 and R¹⁴ is H or lower alkyl;

or R¹ and R² together form a covalent bond;

or R² and R³ together form =Z, where Z is selected from O, S, and NH;

X¹ and X² are each independently selected from the group consisting of—C(R¹⁵)(R¹⁶)—, O, S, NH, C═O, C═S, C═.NH, SO, and SO₂, wherein R¹⁵ and R¹⁶ are each independently selected from H, lower alkyl, hydroxy, lower alkoxy, halo, amino, aminoalkyl, nitro, heteroaryl, aryl, OC(═O)R¹⁷, OC(═O)OR¹⁷, OC(═O)N(R¹⁷)₂, O(CH₂)ₘN(R¹⁷)₂2, C(═O)N(R¹⁷)₂, and O(CH₂)ₘCOOH, where m is 1-5 and R¹⁷ is H or lower alkyl;

or X¹ and X² together form —C═C—;

X³ is selected from the group consisting of O, S, NH, and (CH₂)ₚ where p is 1-3.

or a pharmaceutically acceptable salt thereof.

Suitable tanshinone compounds include those of Formula I:

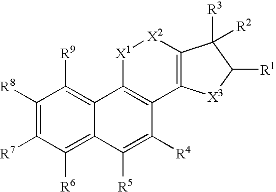

wherein

R¹, R², R³, R⁴, R⁵, R⁶, R⁷, R⁸, and ⁹ are each independently selected from H, lower alkyl, hydroxy, lower alkoxy, halo, amino, aminoalkyl, nitro, heteroaryl, aryl, OC(═O)R¹⁴, OC(═O)OR¹⁴, OC(═O)N(R¹⁴)₂, O(CH₂)ₘN(R¹⁴)₂, C(═O)N(R¹⁴)₂, and O(CH₂)ₘCOOH, where m is 1-5 and R¹⁴ is H or lower alkyl;

or R¹ and R² together form a covalent bond;

or R² and R³ together form =Z, where Z is selected from O, S, and NH;

X¹ and X² are C═O; and

X³ is O;

or a pharmaceutically acceptable salt thereof.

In some embodiments, a suitable compound is 15,16-dihydrotanshinone, which has the structure:

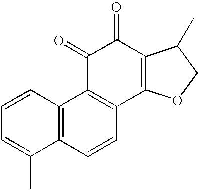

Upon reading the present specification, the ordinarily skilled artisan will appreciate that the pharmaceutical compositions comprising a tanshinone compound described herein can be provided in a wide variety of formulations. More particularly, the tanshinone compound can be formulated into pharmaceutical compositions by combination with appropriate, pharmaceutically acceptable carriers or diluents, and may be formulated into preparations in solid, semi-solid (e.g., gel), liquid or gaseous forms, such as tablets, capsules, powders, granules, ointments, solutions, suppositories, injections, inhalants and aerosols. Where the tanshinone compound is a naturally-occurring compound, the pharmaceutical composition can also be provided as an herbal preparation.

The tanshinone compound formulation used will vary according to the condition or disease to be treated, the route of administration, the amount of tanshinone compound to be administered, and other variables that will be readily appreciated by the ordinarily skilled artisan. In general, and as discussed in more detail below, administration of tanshinone compound can be either systemic or local, and can be achieved in various ways, including, but not necessarily limited to, administration by a route that is oral, parenteral, intravenous, intra-arterial, inter-pericardial, intramuscular, intraperitoneal, intra-articular, intra-ocular, topical, intratumoral, peritumoral, transdermal, transcutaneous, subdermal, intradermal, intrapulmonary, etc.

Where the tanshinone compound is to be delivered to the eye (e.g., for treatment of age-related macular degeneration and other ocular disorders associated with pathological angiogeneisis), the tanshinone compound can be administered by injection (e.g., intraocular (e.g., intravitreal), subcutaneous, or intravenous injection). Other routes of administration include topical administration (to the eye, e.g., using eye drops), subconjunctival, perioocular, subtenon, retrobulbar (retro-orbital), or by iontophoretic delivery to the eye.

In pharmaceutical dosage forms, the tanshinone compound may be administered in the form of their pharmaceutically acceptable salts, or they may also be used alone or in appropriate association, as well as in combination, with other pharmaceutically active compounds. The following methods and excipients are merely exemplary and are in no way limiting.

The tanshinone compound can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Formulations suitable for topical, transcutaneous, and transdermal administration may be similarly prepared through use of appropriate suspending agents, solubilizers, thickening agents, stabilizers, and preservatives. Topical formulations may be also utilized with a means to provide continuous administration of 15,16-dihydrotanshinone or other tanshinone compound by, for example, incorporation into slow-release pellets or controlled-release patches.

In some embodiments, an active agent (e.g., a tanshinone compound) is delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of active agent can be accomplished using any of a variety of refillable, pump systems. Pumps provide consistent, controlled release over time. In some embodiments, the agent is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are used in some embodiments because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present invention. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted infra, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, an active agent is delivered using an implantable drug delivery system, e.g., a system that is programmable to provide for administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the Synchromed infusion pump (Medtronic).

The tanshinone compound can also be formulated in a biocompatible gel, which gel can be applied topically or implanted (e.g., to provide for sustained release of tanshinone compound at an internal treatment site). Suitable gels and methods for formulating a desired compound for delivery using the gel are well known in the art (see, e.g., U.S. Pat. Nos. 5,801,033; 5,827,937; 5,700,848; and MATRIGEL™).

For oral preparations, the tanshinone compound can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The tanshinone compound can be utilized in aerosol formulation to be administered via inhalation. The compounds of the present invention can be formulated into pressurized acceptable propellants such as dichlorodifluoromethane, propane, nitrogen and the like.

Furthermore, the tanshinone compound can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the tanshinone compound in a composition as a solution in sterile water, normal saline or another pharmaceutically acceptable carrier.

The term unit dosage form, as used herein, refers to physically discrete units suitable as unitary dosages for human and/or animal subjects, each unit containing a predetermined quantity of tanshinone compound calculated in an amount sufficient to produce the desired reduction in angiogenesis in association with a pharmaceutically acceptable diluent, carrier or vehicle. The specifications for the unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, a tanshinone compound is administered in a combination therapy with one or more additional therapeutic agents, as discussed. Exemplary therapeutic agents include therapeutic agents used to treat cancer, atherosclerosis, proliferative retinopathies, chronic arthritis, psoriasis, hemangiomas, etc.

### Doses, Dosage Regimen, and Dosage Forms

Tanshinone compound is administered to a subject, e.g., a human, in the context of the present invention in an amount sufficient to effect a therapeutic reduction in angiogenesis in the subject, especially over a reasonable time frame. The dose will be determined by, among other considerations, the potency of the particular tanshinone compound employed, the formulation used, and the condition of the subject, as well as the body weight of the subject to be treated. The dose also will be determined by the existence, nature, and extent of any adverse side-effects that might accompany the administration of a particular compound.

In determining the effective amount of tanshinone compound in the reduction of angiogenesis, the route of administration, the kinetics of the release system (e.g., pill, gel or other matrix), and the potency of the tanshinone compound are considered so as to achieve the desired anti-angiogenic effect with minimal adverse side effects. The tanshinone compound will typically be administered to the subject being treated for a time period ranging from a day to a few weeks, consistent with the clinical condition of the treated subject.

As will be readily apparent to the ordinarily skilled artisan, the dose and dosage regimen is adjusted for tanshinone compound according to their potency and/or efficacy relative to a standard, e.g., 15,16-dihydrotanshinone. A dose may be in the range of about 0.01 mg to 10 mg, given 1 to 20 times daily, and can be up to a total daily dose of about 0.1 mg to 100 mg. If applied topically, for the purpose of a systemic effect, the patch or cream would be designed to provide for systemic delivery of a dose in the range of about 0.01 mg to 10 mg. If the purpose of the topical formulation (e.g., cream) is to provide a local anti-angiogenic effect, the dose would likely be in the range of about 0.001 mg to 1 mg. If injected for the purpose of a systemic effect, the matrix in which the tanshinone compound is administered is designed to provide for a systemic delivery of a dose in the range of about 0.001 mg to 1 mg. If injected for the purpose of a local effect, the matrix is designed to release locally an amount of tanshinone compound in the range of about 0.003 mg to 1 mg.

Regardless of the route of administration, the dose of tanshinone compound can be administered over any appropriate time period, e.g., over the course of 1 to 24 hours, over one to several days, etc. Furthermore, multiple doses can be administered over a selected time period. A suitable dose can be administered in suitable subdoses per day, particularly in a prophylactic regimen. The precise treatment level will be dependent upon the response of the subject being treated.

### Delivery Systems

The present invention further provides a delivery system for delivering a tanshinone compound, e.g., a formulation comprising a tanshinone compound, to an individual in need thereof. Delivery systems include oral delivery systems, and injection systems.

In some embodiments, an active agent (e.g., a tanshinone compound) is packaged for oral administration. The present invention provides a packaging unit comprising daily dosage units of an active agent. For example, the packaging unit is in some embodiments a conventional blister pack or any other form that includes tablets, pills, and the like. The blister pack will contain the appropriate number of unit dosage forms, in a sealed blister pack with a cardboard, paperboard, foil, or plastic backing, and enclosed in a suitable cover. Each blister container may be numbered or otherwise labeled, e.g., starting with day 1.

In some embodiments, a subject delivery system comprises an injection device. Exemplary, non-limiting drug delivery devices include injections devices, such as pen injectors, and needle/syringe devices. In some embodiments, the invention provides an injection delivery device that is pre-loaded with a formulation comprising an effective amount of an active agent (e.g., a tanshinone compound). For example, a subject delivery device comprises an injection device pre-loaded with a single dose of an active agent (e.g., a tanshinone compound). A subject injection device can be re-usable or disposable.

Pen injectors are well known in the art. Exemplary devices which can be adapted for use in the present methods are any of a variety of pen injectors from Becton Dickinson, e.g., BD™ Pen, BD™ Pen II, BD™ Auto-Injector; a pen injector from Innoject, Inc.; any of the medication delivery pen devices discussed in U.S. Pat. Nos. 5,728,074, 6,096,010, 6,146,361, 6,248,095, 6,277,099, and 6,221,053; and the like. The medication delivery pen can be disposable, or reusable and refillable.

### Subjects Suitable for Treatment

Subjects suitable for treatment (“individuals in need of treatment”) using the methods of the instant invention include a subject who has a condition or disorder described above, e.g., a disorder amenable to treatment by reducing angiogenesis. Subjects suitable for treatment include individuals having a tumor, where there is a risk that cancer cells in the tumor will metastasize; individuals having an ocular disorder resulting from pathological angiogenesis, e.g., individuals having age-related macular degeneration, individuals having wet AMD, etc.); individuals suffering from arthritis; and the like, e.g., any individual having a disorder associated with or resulting from pathological angiogenesis, as discussed above.

### EXAMPLES

The following examples are put forth so as to provide those of ordinary skill in the art with a complete disclosure and description of how to make and use the present invention, and are not intended to limit the scope of what is regarded as the invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### 15,16-Dihydrotanshinone Reduces Endothelial Cell Proliferation

An endothelial cell proliferation assay was carried out according to the procedures of Connally, et al. (1986) (Anal. Biochem. 152:136-140) with modifications (Liang and Wong (2000) ANGIOGENESIS: FROM THE MOLECULAR TO INTEGRATIVE PHARMACOLOGY, edited by Maradoudakis, Kluwer Academic/Plenum Publishers, New York, pp 209-223). Bovine Cardio-Pulmonary Artery Endothelial (CPAE) cells acquired from American Type Culture Collection (ATCC; ATCC No. CCL-209) were grown to nearly 95% confluence in MEM-10E. The cells were released from the tissue culture flask with a 0.25% trypsin solution and plated in 24-well tissue culture plates in the same culture medium at a density of 10,000 cells/well. After the plates were incubated for 8 hours at 37° C. in a 5% carbon dioxide incubator, assay samples and control samples were added. Each sample was loaded in two different wells at 100 μl/well to ensure reproducibility. After incubation with the sample for 60 hours, the medium was aspirated, and the number of cells was measured on the basis of the calorimetric measurement of cellular acid phosphatase.

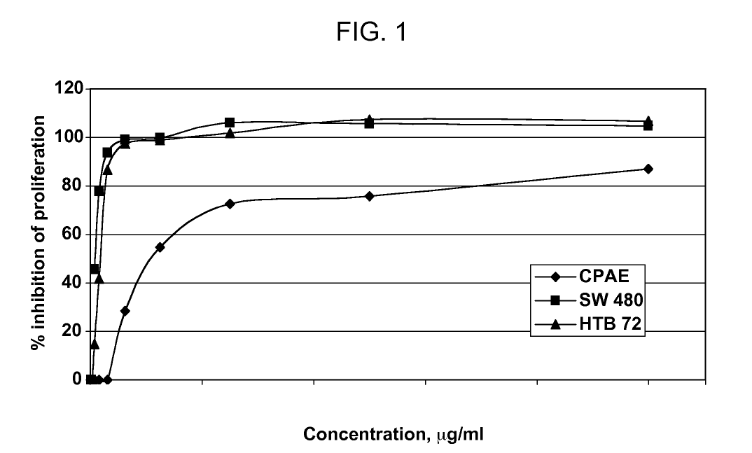

The results showed that 15,16-dihydrotanshinone I inhibits endothelial cell proliferation. At a low concentration of 50 μg/ml, 15,16-dihydrotanshinone I achieves 84.4% of endothelial cell growth inhibition. A concentration-dependent study on the inhibition of endothelial cell proliferation by another 15,16-dihydrotanshinone I sample was performed and the results are shown in FIG. 1. It is clear that at very low concentration, 15,16-dihydrotanshinone I is a very strong inhibitor of endothelial cell proliferation. Furthermore, 15,16-dihydrotanshinone I inhibits endothelial cell proliferation in a concentration-dependent manner.

15,16-dihyrotanshinone I is also a potent inhibitor of several cancer cell lines in vitro, including the SW480 (Colon Cancer) and the HTB 72 (Melanoma) cancer cell lines. At a very low concentration of about 2 micrograms per milliliter, 100% inhibitions are achieved. This suggests the additional metastatic inhibiting action of this compound.

### Example 2

### Treating AMD with a Tanshinone Compound

An individual displaying symptoms of neovascular (“wet”) age-related macular degeneration (AMD) is treated with multiple doses of the tanshione compound at regular intervals. A formulation including a tanshinone compound at concentrations of 50 μg/ml, 100 μg/ml, and 200 μg/ml, for a total dosage of 50 μg, 100 μg, or 200 μg, respectively is administered to the individual by intraocular injection. Vision improvement is determined by asking patients to read a standard letter chart.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of reducing pathological angiogenesis in a mammal in need thereof, the method comprising administering to the mammal a substantially pure tanshinone compound in an amount effective to reduce angiogenesis, and wherein the tanshinone compound is a compound of Formula I 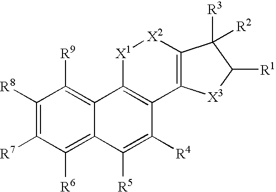
wherein
R¹R², R³, R⁴, R⁵, R⁶, R⁷, R⁸, and ⁹ are each independently selected from H, lower alkyl, hydroxy, lower alkoxy, halo, amino, aminoalkyl, nitro, heteroaryl, aryl, OC(═O)R¹⁴, OC(═O)OR¹⁴, OC(═O)N(R¹⁴)₂, O(CH₂)ₘN(R¹⁴)₂, C(═O)N(R¹⁴)₂, and O(CH₂)ₘCOOH, where m is 1-5 and R¹⁴ is H or lower alkyl;
or R¹ and R² together form a covalent bond;
or R² and R³ together form =Z, where Z is selected from O, S, and NH;
X¹ and X² are C═O; and
X³ is O.

2. The method of claim 1, wherein the tanshinone compound is a compound of the structure: 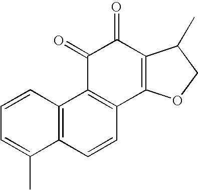
or a pharmaceutically acceptable salt thereof.

3. The method of claim 1, wherein said administering is by a route selected from intravenous, systemic, oral, intraarterial, in or around a joint, topical, intraocular, intravitreous, subconjunctival, periocular, subtenon, and retrobulbar.

4. The method of claim 1, wherein the pathological angiogenesis is associated with cancer cell metastasis, and wherein the tanshinone compound is administered in an amount effective to reduce angiogenesis and reduce metastasis.

5. The method of claim 4, wherein said administering is peri-tumoral.

6. The method of claim 1, wherein the mammal is other than a cancer patient.

7. The method of claim 1, further comprising administering at least one additional anti-angiogenic agent.

8. The method of claim 4, wherein said administering is carried out as an adjuvant to a standard cancer therapy.

9. The method of claim 8, wherein said standard cancer therapy comprises chemotherapy, surgery, radiation therapy, or bone marrow transplantation.

10. A method of treating an arthritic disorder associated with pathological angiogenesis in an individual in need thereof, the method comprising administering to the individual a substantially pure tanshinone compound in an amount effective to reduce pathological angiogenesis and treat the arthritic disorder, wherein the tanshinone compound is a compound of Formula I 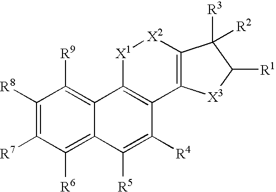
wherein
R¹, R², R³, R⁴, R⁵, R⁶, R⁷, R⁸, and ⁹ are each independently selected from H, lower alkyl, hydroxy, lower alkoxy, halo, amino, aminoalkyl, nitro, heteroaryl, aryl, OC(═O)R¹⁴, OC(═O)OR¹⁴, OC(═O)N(R¹⁴)₂, O(CH₂)ₘN(R¹⁴)₂, C(═O)N(R¹⁴)₂, and O(CH₂)ₘCOOH, where m is 1-5 and R¹⁴ is H or lower alkyl;
or R¹ and R² together form a covalent bond;
or R² and R³ together form =Z, where Z is selected from O, S, and NH;
X¹ and X² are C═O; and
X³ is O.

11. The method of claim 10, wherein the tanshinone compound is a compound of the structure: 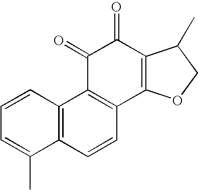
or a pharmaceutically acceptable salt thereof.

12. The method of claim 10, wherein the arthritic disorder is osteoarthritis.

13. The method of claim 10, wherein the arthritic disorder is rheumatoid arthritis.

14. The method of claim 10, wherein the arthritic disorder is psoriatic arthritis.

15. The method of claim 10, wherein said administering is systemic.

16. The method of claim 10, wherein said administering is at or near an arthritic joint.

17. The method of claim 10, wherein said administering is oral.

18. The method of claim 10, further comprising administering at least a second therapeutic agent suitable for treating the arthritic disorder.

19. A method of treating an ocular disorder associated with pathological angiogenesis in an individual in need thereof, the method comprising administering to the individual a substantially pure tanshinone compound in an amount effective to reduce pathological angiogenesis and treat the ocular disorder, wherein the tanshinone compound is a compound of Formula I 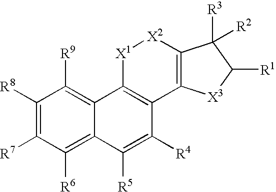
wherein
R¹, R², R³, R⁴, R⁵, R⁶, R⁷, R⁸, and ⁹ are each independently selected from H, lower alkyl, hydroxy, lower alkoxy, halo, amino, aminoalkyl, nitro, heteroaryl, aryl, OC(═O)R¹⁴, OC(═O)OR¹⁴, OC(═O)N(R¹⁴)₂, O(CH₂)ₘN(R¹⁴)₂, C(═O)N(R¹⁴)₂, and O(CH₂)ₘCOOH, where m is 1-5 and R¹⁴ is H or lower alkyl;
or R¹ and R² together form a covalent bond;
or R² and R³ together form =Z, where Z is selected from O, S, and NH;
X¹ and X² are C═O; and
X³ is O.

20. The method of claim 19, wherein the tanshinone compound is a compound of the structure: 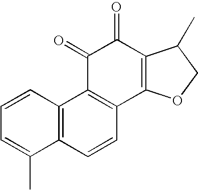
or a pharmaceutically acceptable salt thereof.

21. The method of claim 19, wherein the ocular disorder is diabetic retinopathy.

22. The method of claim 19, wherein the ocular disorder is age-related macular degeneration.

23. The method of claim 19, wherein said administering is intraocular, intravitreous, subconjunctival, periocular, subtenon, retrobulbar, or oral.

24. The method of claim 19, further comprising administering a vascular endothelial growth factor antagonist.
